(19) United States
(12) Patent Application Publication
(10) Pub. No.: US 2008/0119781 A1
(43) Pub. Date: May 22, 2008

(54) Method and apparatus for treating scar tissue

(76) Inventor: Jonathan King, Staten Island, NY (US)

Correspondence Address: Allen R. Kipnes, Esq.;WATOV & KIPNES, P.C., P.O. Box 247, Princeton Junction, NJ, 08550, US

(21) Appl. No.: 11/603,684

(22) Filed: Nov. 22, 2006

(51) Int. Cl. A61B 17/20; A61K 31/135; A61B 17/34; A61P 17/02

(52) U.S. Cl. 604/22; 514/653; 606/185

(57) ABSTRACT

A method for treating an area of the skin containing scar tissue is provided to include the steps of perforating the area of the skin containing the scar tissue with at least one perforation to initiate a wound healing response therein, and simultaneously introducing a scar treating liquid to the perforation for diffusion into the scar tissue. The present invention is further directed to an apparatus for implementing the method.

### FIELD OF THE INVENTION

The present invention relates generally to the treatment of scar tissue, and more particularly to a method and an apparatus useful for removing or at least substantially diminishing the presence of scar tissue in the skin.

### BACKGROUND OF THE INVENTION

Scar tissues are a natural by-product of the biological process of wound repair in the skin and other tissues of the body. With the exception of very minor lesions, every wound caused by accident, disease, or surgery, results in some degree of scarring. Scar tissue typically develops when the deeper, thick dermis region of the skin is damaged. The body forms new collagen fibers to mend the damage, resulting in a scar. The new scar tissue typically exhibits an appearance, texture and quality that are different from the surrounding healthy normal tissue.

There are several different kinds of scar tissues. Most scar tissues are flat and pale and leave only a trace of the original injury. Other scar tissues have a raised appearance when the body has produced too much collagen. Such raised scar tissues include hypertropic scars and keloid scars. Another type exhibits a sunken or pitted appearance. This appearance occurs when underlying structures supporting the skin (for example, fat or muscle) are lost or compromised. The formation of scar tissue is influenced by several factors including the individual's age, cause of injury and location of the injury on the body. Younger skin typically makes strong repairs and tends to overheal, resulting in larger, thicker scar tissues than those in older skin.

Scar tissues are typically inferior in terms of cosmetic and functional quality as compared to the normal original tissue. Scar tissues lack suppleness and elasticity, appear unsightly, and are less resistant to ultraviolet radiation. For these reasons, scar tissues are undesirable. Numerous scar treating techniques have been developed including surgical treatment, dermal coverings, pressure treatments, dermabrasion, topical treatments such as cocoa butter, vitamin E, oils, greases, and the like, wound dressings, collagen implantation, cortisone injections, laser ablation, grafts and excisions, chemical peels, and cryosurgery. Such techniques can only be used to improve the way scars look by changing the size, depth or color. Results from such techniques vary widely, and often require regular applications of the scar treating techniques to maintain acceptable results and appearance.

Accordingly, there is a need for a method and an apparatus capable of treating scar tissue. There is a need for a method and an apparatus designed to greatly improve the appearance of the skin afflicted with scar tissue, and revitalize the surrounding healthy normal tissue. There is a further need for a method and an apparatus useful for removing or at least substantially diminishing the presence of scar tissue, while effectively promoting a desirable wound healing response that minimizes the reformation of the scar tissue.

### SUMMARY OF THE INVENTION

The present invention relates generally to a method and an apparatus useful for treating scar tissue. The method and apparatus are designed to remove or at least substantially diminish the presence of the scar tissue from an area of the skin. The method of the present invention generally involves forming at least one perforation in the area of the skin containing the scar tissue to initiate a wound healing response, while simultaneously introducing a scar treating liquid through the perforation for diffusion into the perforated scar tissue. The perforation is preferably made at a sufficient depth to induce growth of normal tissue.

In particular, the method comprises perforating the area of the skin containing scar tissue to produce a plurality of perforations, while simultaneously introducing a scar treating liquid through the perforations for diffusion into the perforated scar tissue. The method of the present invention is implemented through an apparatus of the present invention utilizing dermal perforation techniques of the present invention to remove or at least substantially diminish the presence of scar tissue, while effectively stimulating the skin's wound healing response.

In accordance with the present invention, the apparatus of the present invention is adapted to produce at least one perforation in an area of the skin containing scar tissue, and to simultaneously introduce a scar treating liquid through the perforation for diffusion into the perforated scar tissue. The apparatus is further designed to perforate the skin at a pre-determined depth in a rapid and precise manner. The treated scar tissue is thereafter allowed to heal over time, resulting in the at least a substantial elimination of the scar tissue. The method and apparatus of the present invention are relatively straightforward and cost effective to implement.

In one aspect of the present invention, there is provided a method for treating an area of the skin containing scar tissue, which comprises:

perforating the area of the skin containing the scar tissue with at least one perforation to initiate a wound healing response therein; and

simultaneously introducing a scar treating liquid to the perforation for diffusion into the scar tissue.

In another aspect of the present invention, there is provided an apparatus for treating an area of the skin containing scar tissue, which comprises:

a needle bar comprising at least one needle, the at least one needle adapted for perforating the area of the skin containing scar tissue to yield at least one perforation;

oscillating means for oscillating the bar to axially move the at least one needle in a reciprocating motion; and

scar treating liquid supply means associated with the at least one needle for supplying a scar treating liquid to the perforation for diffusion into the scar tissue.

### BRIEF DESCRIPTION OF THE DRAWINGS

The following drawings are illustrative of embodiments of the present invention and are not intended to limit the invention as encompassed by the claims forming part of the application.

FIG. 1 is a perspective view of an apparatus for treating scar tissue for one embodiment of the present invention;

FIG. 2 is an exploded assembly view of a hand-holdable dermal perfusion handpiece of the apparatus of FIG. 1 in accordance with the present invention;

FIG. 3 is a front elevation view of the dermal perfusion handpiece of the apparatus in accordance with the present invention;

FIG. 4 is a top plan view of the dermal perfusion handpiece of the apparatus in accordance with the present invention;

FIG. 5 is a side cross sectional view of portions of the dermal perfusion handpiece comprising a needle bar assembly and a hand-holdable tubular housing in accordance with the present invention;

FIG. 6 is an elevational view of a needle bar assembly for one embodiment of the present invention; and

FIG. 7 is a side elevational view of the needle bar assembly shown in FIG. 6 in accordance with the present invention.

### DETAILED DESCRIPTION OF THE INVENTION

The present invention relates generally to a method and an apparatus useful for treating scar tissue. The method and apparatus are designed to remove or at least substantially diminish the presence of the scar tissue from an area of the skin. The method of the present invention generally involves forming at least one perforation in the area of the skin containing the scar tissue to initiate a wound healing response, while simultaneously introducing a scar treating liquid to the perforation for diffusion into the perforated scar tissue. The perforation is preferably made at a sufficient depth to induce a wound healing response and corresponding growth of normal tissue.

In particular, the method comprises perforating the area of the skin containing scar tissue to produce a plurality of perforations, while simultaneously introducing a scar treating liquid to the perforations for diffusion into the perforated scar tissue. More preferably, the perforations are formed at select points within and/or around the scar tissue at a depth within the dermis region of the skin, while minimally affecting the surrounding healthy normal tissue. The method of the present invention is implemented through an apparatus of the present invention utilizing dermal perfusion techniques of the present invention to remove or at least substantially diminish the presence of scar tissue, while effectively stimulating the skin's wound healing response.

In accordance with the present invention, the apparatus of the present invention is adapted to produce at least one perforation in an area of the skin containing scar tissue, and to simultaneously introduce a scar treating liquid to the perforation for diffusion into the perforated scar tissue. The apparatus is further designed to perforate the skin at a pre-determined depth in a rapid and precise manner. The treated scar tissue is thereafter allowed to heal over time, resulting in the at least a substantial elimination of the scar tissue. The method and apparatus of the present invention are relatively straightforward and cost effective to implement.

The method and apparatus of the present invention are intended to treat any scar tissue present in the bodies of warm-blooded animals including humans. More specifically, the present method and apparatus are designed to treat scar tissue in the skin including keloid scars, hypertrophic scars, and combinations thereof.

The process of wound healing is complex. Cytokines released by the vascular endothelial cells and epidermal keratinocytes are responsible for initiating the increased production of the dermal extracellular matrix (ECM). These elements lie in the uppermost regions of the skin. This ECM production process takes place in a series of interrelated steps by the resident cells of the dermis.

The method and apparatus of the present invention provide an effective treatment of scar tissue. Applicant believes that the method and apparatus of the present invention functions to promote the break-up and absorption of the scar tissue, while promoting a wound healing response in the dermis region of the skin. This wound healing response promotes growth of the dermal extracellular matrix (ECM) in the upper and lower dermis region. The method and apparatus of the present invention functions to concentrate the wound healing response close to the dermis region of the skin, and accordingly close to the location of the scar tissue being treated.

In one form of the present invention, there is provided a method for treating an area of the skin containing scar tissue, wherein the method comprises perforating the area of the skin containing the scar tissue with at least one perforation to initiate a wound healing response therein, while simultaneously introducing a scar treating liquid to the perforation for diffusion into the scar tissue.

The method may be implemented by perforating the area of the skin containing the scar tissue to produce multiple perforations extending down to the dermis layer of the skin in the presence of a scar treating liquid. The perforations in the skin area are generally produced at a depth beneath the epidermis region of the skin, preferably in the upper to lower dermal region of the skin. Preferably, the perforations in the skin area are produced at a depth of at least 0.4 mm from the surface of the scar tissue, and more preferably at a depth of from about 0.5 mm to 5 mm from the surface of the scar tissue. The perforations formed are microscopic in size, typically on a micron scale.

As used herein, the term “scar treating liquid” is intended to be broad enough to encompass any bio-compatible liquid that substantially lacks any permanent pigmentation agents. The term “permanent pigmentation agents” encompasses any pigments used in cosmetic tattooing, micropigmentation, and dermapigmentation that are formulated for insertion into skin's dermal layer to permanently impart color to the skin distinct from the existing pigmentation. Preferably, the scar treating liquid is bacteria free, sterilized distilled water. By way of example, the scar treating liquid may be selected from saline solutions including normal saline, half-normal saline, quarter-normal saline, Ringer's solution, solution of 5% dextrose in water (D5W) and the like.

It is believed that the perfusion of the scar treating liquid into the perforated scar tissue helps to maintain proper hydration, while promoting the growth of normal skin cells, which ultimately replaces the scar tissue with normal tissue, and further ensures that the perforations in the skin remains substantially clean and sterile during the treatment and subsequent healing process. The scar treating liquid may be administered alone or in combination with a therapeutic agent. The therapeutic agent is preferably an agent capable of promoting wound healing while minimizing scarring, and may be selected from antibacterial agents, antioxidants, enzymes, growth hormones, anti-inflammatory agents, growth factors, and combinations thereof.

After each round of treatment, healing time and care will depend on the size and depth of the treated area of the skin, the location of the treated skin, age and condition of the subject. Generally, the healing time ranges from about one or two days to several weeks, and may extend to a time period of from about six to eight weeks. It is recommended that the treated skin area be maintained in an occluded condition to retain the skin's moisture and stimulate wound healing. A coating of a petroleum-based ointment such as petrolatum or petroleum jelly, or a moisturizing lotion, may be used for occluding the treated skin area to confine moisture to the skin. The petroleum-based ointment or moisturizing lotion may further comprise a vasoconstrictor such as phenylephrine, to substantially minimize bleeding of blood, serum and other exudates from the treated skin area. The term “vasoconstrictor” includes any agent or drug, which can act to cause the constriction of blood vessels. A suitable ointment containing phenylephrine is Preparation H®, manufactured by Wyeth of Madison, N.J.

Preferably, after treatment, the treated area of the skin is covered with a pharmaceutical-grade petroleum-based ointment such as white petrolatum or petroleum jelly, and observed for any exudate or droplet formation on the surface of the treated skin. If the exudates or droplets that emerge exhibit color such as pink, the droplets are carefully absorbed with an absorbent material, or lightly rubbed periodically to prevent platelet and dead cell buildup. This process is repeated until the exudates or droplets become substantially colorless, and the treated area is clear.

Afterward, the treated area is provided with a coating of an ointment sufficiently to occlude the skin. A plastic wrap such as cellophane film, cling film, and the like, or any suitable occlusive bandage, may be applied over the ointment layer where the treated area of the skin may be exposed to irritants or foreign matter. Washing of the treated area is employed where dead cells or foreign matter have accumulated on the surface and application of the ointment is repeated thereafter.

It is preferable to apply a dressing comprising the ointment and plastic wrap soon after treatment. After about an hour, the dressing is removed from the treated area and cleaned with mild soap and water and a new dressing is thereafter applied.

The term “treatment” is used herein to mean that, at a minimum, administration of a method of the present invention mitigates dermal scar tissue in warm-blooded animals, including humans. Thus, the term “treatment” includes at least substantially removing, alleviating and/or reversing scarring or the presence of scar tissue in epidermis and dermis regions of the skin. Depending on the severity and condition of the scar tissue, the treatment may require multiple rounds or administrations of the method of the present invention.

Generally, the specific number of rounds of treatment of the present invention to be administered, as well as the duration of treatment, is mutually dependent. The number of rounds and treatment regimen will also depend upon such factors as the size of the skin area containing scar tissue, the type of scar tissue involved, the part of the body where the scar tissue is present in the skin, the age of the scar, the nature and extent of other scarring, the personal attributes of the subject (such as weight, age, and the like), compliance with the treatment regimen, and the presence and severity of any possible side effects of the treatment.

For administration of the method of the present invention, typically for a human adult, at least one round of treatment may be necessary to reduce or mitigate the presence of the scar tissue in the area of the skin to be treated. Preferably, the number of rounds of treatment may be at least two to three, and more preferably from about 2 to 8 rounds of treatment spaced over a period of a day or two to several weeks. The foregoing notwithstanding, it should be fully understood that the number of rounds of treatment and treatment duration set forth herein are exemplary only and they do not, to any extent, limit the scope of practice of the present invention.

The method of the present invention is implemented through the use of an apparatus of the present invention for the removal, alleviation and/or reversal of at least a substantial portion the scar tissue in the area of the skin to be treated as will now be described.

In another form of the present invention, there is provided an apparatus for treating an area of the skin containing scar tissue, which comprises a needle bar comprising at least one needle, the at least one needle adapted for perforating the area of the skin containing scar tissue to yield at least one perforation, oscillating means for oscillating the bar to axially move the at least one needle in a reciprocating motion, and scar treating liquid supply means associated with the at least one needle for supplying a scar treating liquid to the perforation for diffusion into the scar tissue.

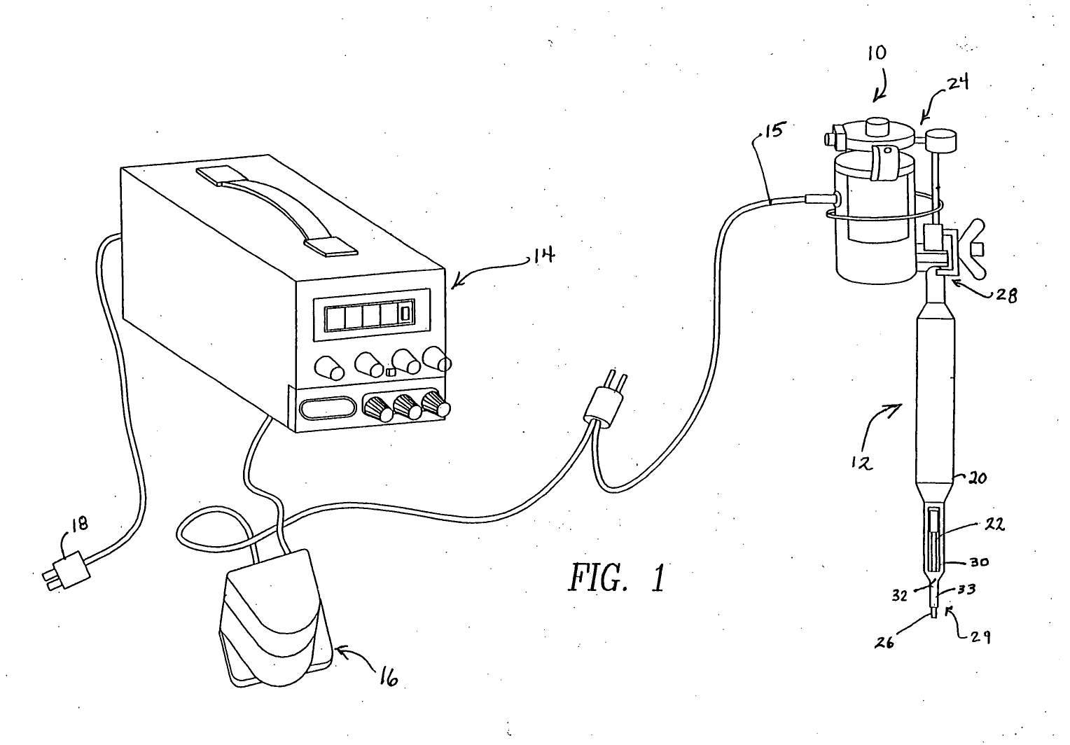

Referring to FIG. 1, there is shown a scar treating apparatus, which is designated by the reference numeral 10. The apparatus 10 includes a handpiece 12 electrically connected to a power pack 14 via power line 15 with a foot switch 16. The power pack 14 is a serial control type regulated DC power source. The power pack 14 is constructed to supply adjustable DC electrical power to the handpiece 12 after converting power from an alternating current power source (not shown) connected via plug 18. The operator may vary the voltage and current output of the power pack 14 as needed. The foot switch 16 is a biased switch, and includes a pair of contacts (not shown) that is maintained in a normally open state by a spring, which prevents electricity from flowing through the power line 15. In this manner, power to the handpiece 12 is supplied when the foot switch 16 is depressed, and power is shut off when the foot switch 16 is released.

The handpiece 12 includes a hand-holdable tubular housing 20, a needle assembly 22 located within the housing 20, and a reciprocating assembly 24 operatively associated with the needle assembly 22. The housing 20 is open at both ends 28 and 29. A portion of the needle assembly 22 extends through the open end 28 of the housing 20, and is attached to the reciprocating assembly 24. The needle assembly 22, driven by the reciprocating assembly 24, moves back and forth in the housing 20 to produce a reciprocating slide motion. The housing 20 includes a window opening 30 to facilitate cleaning and inspection of the needle assembly 22 during use, and a needle guide portion 33 for ensuring proper positioning of the needle assembly 22 during use.

The needle assembly 22 includes a needle tip portion 26, which includes at least one sterile needle 56 (shown best in FIG. 6). The size of the needle 56 may encompass any medically accepted size suitable for making microscopic perforations in the skin to achieve the objective of the present invention. The needle size is about less than 0.54 mm, and preferably from about 0.25 mm to 0.35 mm. For lighter scarring of the skin, a smaller needle size is used to treat the skin area. For heavier thicker scars, a larger needle size may be necessary.

The needle tip portion 26 extends through the open end 29 of the housing 20 at a length corresponding to the depth of the perforations in the skin formed therefrom. During operation, the needle tip portion 26 is adapted to reciprocate back and forth at a rate of from about 50 to 3000 cycles per minute. The needle tip portion 26 is adapted to pierce through the surface of the scar tissue in the skin and produce microscopic perforations. The needle tip portion 26 is moved about by the operator to produce perforations over the entire surface of the scar tissue at a predetermined depth and density. The housing 20 further includes a reservoir 32 for holding and supplying a scar treating liquid to the needle tip portions 26 as will be described hereinafter.

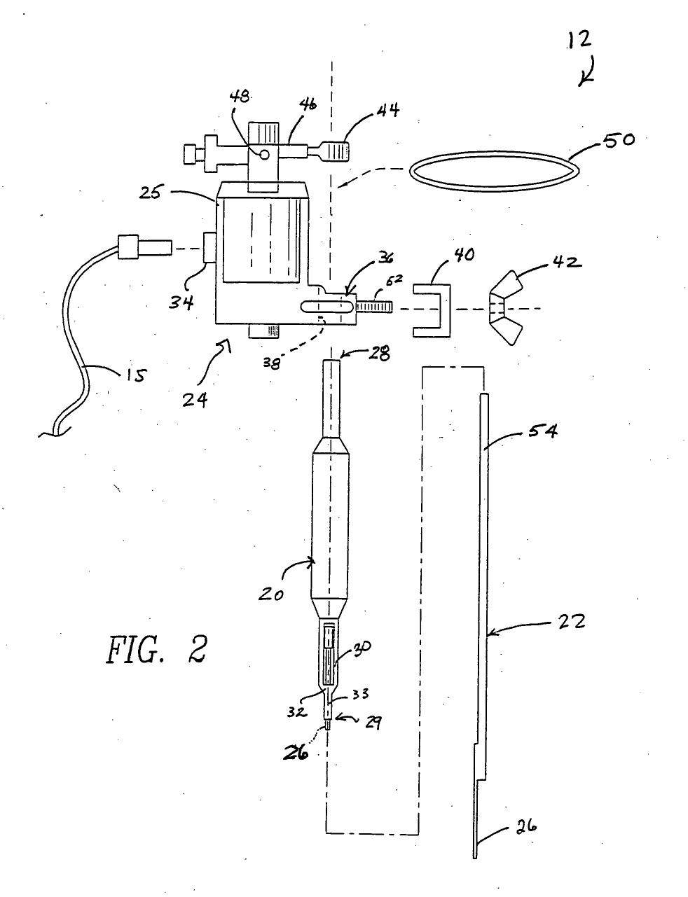

Referring to FIG. 2, the reciprocating assembly 24 includes a motor unit 25, a mounting bracket assembly 36 having a bore 38, a retaining clip 40, a threaded spindle 52 and a fastener 42, an oscillating arm 46 mounted on a pivot bracket 48, a needle bar holder 44 extending from the oscillating arm 46, an elastic band 50, and a power socket 34 configured for receiving and retaining the power line 15. The end 28 of the housing 20 is inserted through the bore 38 of the mounting bracket assembly 36 and held in position by the retaining clip 40. The fastener 42 is engaged to the threaded spindle 52 to maintain the retaining clip 40 in position.

The needle assembly 22 includes a needle bar 54 on which the needle tip portion 26 is mounted. It is preferred that the needle bar 54 is a 15- or 16-gauge size. The needle assembly 22 is inserted through the interior portion of the housing 20 with the needle tip portion 26 extending through the end 29 of the housing 20. The distal end of the needle bar 54 is fixedly attached to the needle bar holder 44 of the reciprocating assembly 24. The elastic band 50 extends snugly around the mid-portions of the reciprocating assembly 24 and the needle bar 54.

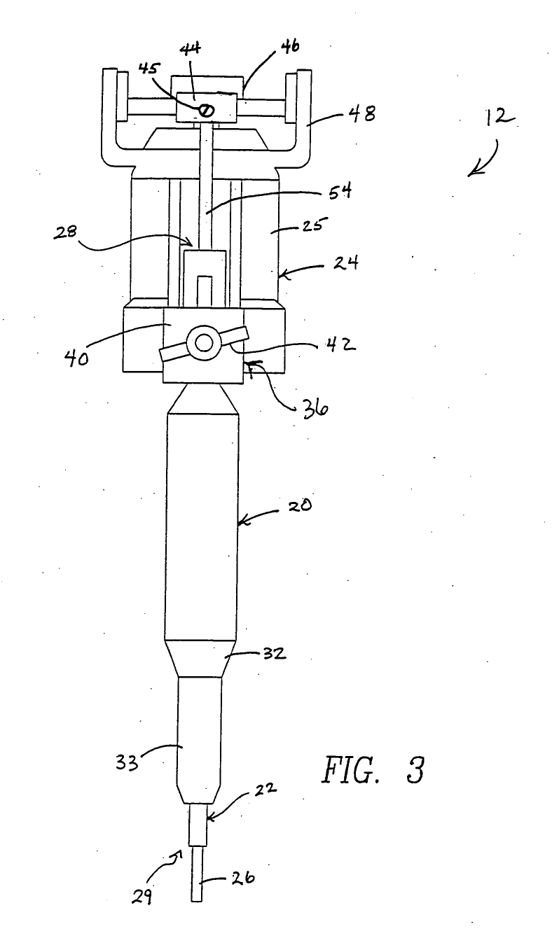

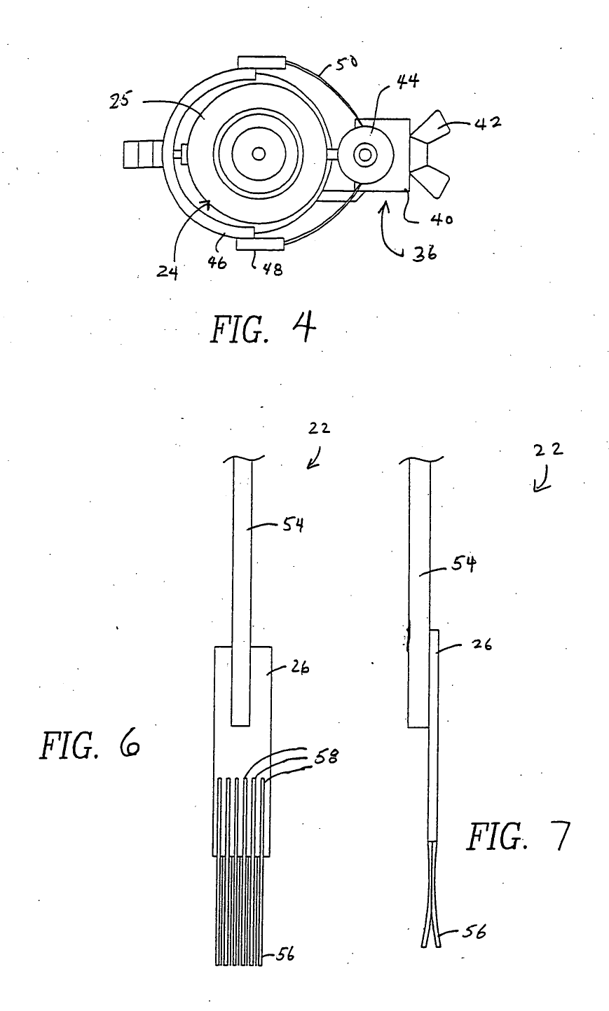

Referring to FIGS. 3 and 4, the housing 20 is fixedly attached to the mounting bracket assembly 36 of the reciprocating assembly 24. The needle bar 54 of the needle bar assembly 22 is attached to the needle bar holder 44 of the oscillating arm 46. The motor unit 25 drives a cam (not shown), which actuates a reciprocating drive rod (not shown). The reciprocating drive rod (not shown) is operatively engaged to the oscillating arm 46 mounted on the pivot bracket 48. In this manner, the reciprocating drive rod (not shown) driven by the motor unit 25 moves in a back-forth motion during operation, thus causing the oscillating arm 46 to move the needle bar holder 44 and needle bar 54 in a vertically reciprocating motion in such a manner that no serious shaking is produced in operation, and the holding of the present apparatus 10 is comfortable and relatively effortless. The needle tip portion 26 slidably mounted in the needle guide portion 33 of the housing 20, reciprocates in accord with the movement of the oscillating arm 46 and the needle bar 54. As noted, the length of the needle tip portion 26 extending from the end 29 of the housing 20 corresponds to the perforation depth of the skin being treated.

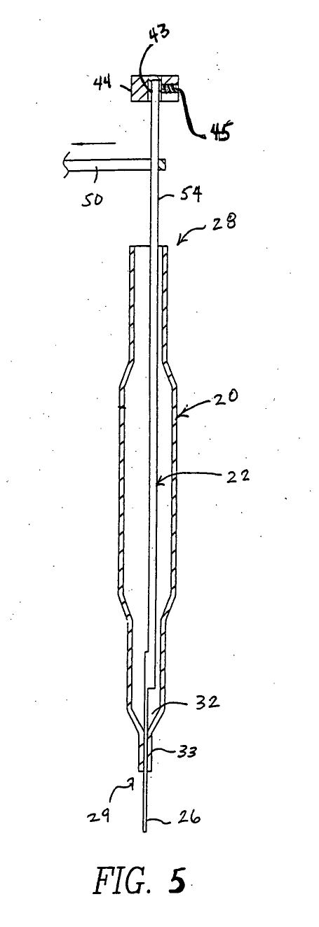

Referring to FIG. 5, the needle tip portion 26 is firmly seated in the needle guide portion 33 of the housing 20 by the elastic band 50. The elastic band 50 pulls the needle bar 54 against the backside of the needle guide portion 33 of the housing 20 to ensure uniform and consistent needle motion during use without excessive shaking. The reservoir 32 is adapted to hold and supply a scar treating liquid to the needle tip portion 26 with each oscillating movement. The reservoir 32 may be refilled periodically during use through the window 30 (as shown best in FIG. 1).

The needle bar holder 44 is configured to securely hold the needle bar 54 in a rigidly fixed configuration to provide precise and uniform perforations in the area of the skin being treated. The needle bar holder 44 enhances the vertical movement of the needle bar 54 for more predictable operation, and substantially minimizes or eliminates any undesirable lateral movement of the needle bar 54, which may adversely affect uniformity in the character and depth of the perforations in the area of the skin being treated.

In a preferred embodiment of the present invention, the needle bar holder 44 includes a bore 43 for receiving and securely retaining the end of the needle bar 54, and a set screw fastener 45 located in a side portion thereof. The bore 43 is machined to receive the end of the needle bar 54 under tight mechanical tolerances. Upon inserting the end of the needle bar 54 into the holder bore 43, the set screw fastener 45 is screwed into the needle bar holder 44 to apply pressure against the end of the needle bar 54 residing in the bore 43 of the holder 44. In this manner, secure retainment and prevention of any unsteady lateral movement in the needle bar 54 during operation is achieved. The screw fastener 45 may be unscrewed to replace the needle bar 54.

Referring to FIGS. 6 and 7, the needle bar assembly 22 is constructed by attaching the needle tip portion 26 to the needle bar 54 through any suitable means including welding, adhesives, and the like. The needle tip portion 26 further includes one or more small needles 56 mounted in substantially axial alignment, and liquid conveying grooves 58 in communication with the needles 56. The small needles 56 are aligned side-by-side in line abreast and soldered together with adjacent needles closely spaced together. The density of the perforations produced in the skin by the handpiece 12 may be selected based on the spacing and arrangement of the small needles 56 on the needle tip portion 26.

It will be understood that different needle configurations may be used depending on the condition, shape, size and type of scar tissue present. The grooves 58 are adapted to receive the scar treating liquid when the needle tip portion 26 is retracted into the housing 20 during the up stroke, and the grooves 58 come in contact with the scar treating liquid retained in the reservoir 32. The scar treating liquid flows along the grooves 58 to the ends of the needles 56 and is deposited into the corresponding perforations of the scar tissue during use.

With reference to FIGS. 1-7, the overall operation of the scar treating apparatus 10 will be described hereinafter. The user holds the handpiece 20 by the tubular housing 20. The power pack 14 is switched on, and the current and voltage is adjusted to provide a desired reciprocating cycle speed of the needle tip portion 26. The reciprocating cycle speed may be varied from 50 to 3000 cycles per minute. The position of the housing 20 with respect to the needle bar assembly 22 can be adjusted to select the depth of the perforations to be administered by the apparatus 10. To select the perforation depth, the fastener 42 may be loosened to move the housing 20 vertically back and forth to adjust the length of the needle tip portion 26 extending through the housing end 29. The perforation depth can be adjusted to a depth of at least 0.4 mm, and preferably from about 0.5 mm to 5 mm.

The scar treating liquid is added through the window opening 30 into the reservoir 32 of the housing 20. The user holds the handpiece 12 by the end 29 of the tubular housing 20 in a manner similar to holding a writing instrument. The handpiece 12 may be oriented at an angle of from about 30° to 90° to the surface of the treated skin. The needle tip portion 26 is placed against the surface of the skin containing the scar tissue. The foot switch 16 activates the handpiece 12 upon depression by the user's foot.

The needle bar assembly 22 driven by the reciprocating assembly 24 causes the needle tip portion 26 to reciprocate back and forth against the skin. The reciprocating action of the needle tip portion 26 conveys precise amounts of the scar treating liquid along the grooves 58 from the reservoir 33 to the needles 56. The needles 56 are pushed into the skin and thereby produce microscopic perforations extending down to the epidermal and dermal regions of the skin. As the needles 56 extend down to the epidermal and dermal regions, small amounts of the scar treating liquid are pushed or diffused into the scar tissue. This process is repeated as the user guides the needle tip portion 26 over the surface of the scar tissue. Once treatment is completed, the treated skin is maintained in a clean, sanitary condition as previously described until the skin is healed.

The foregoing discussion discloses and describes merely exemplary embodiments of the present invention. One skilled in the art will readily recognize from such discussion, and from the accompanying drawings and claims, that various changes, modifications and variations can be made therein without departing from the spirit and scope of the invention as defined in the following claims.

What is claimed is:

1. A method for treating an area of the skin containing scar tissue, comprising:
perforating the area of the skin containing the scar tissue with at least one perforation to initiate a wound healing response therein; and
simultaneously introducing a scar treating liquid to the perforation for diffusion into the scar tissue.

2. The method of claim 1 wherein the area of the skin containing the scar tissue is perforated at a sufficient depth to induce growth of normal tissue.

3. The method of claim 2 wherein the depth is at least 0.4 mm from the surface of the scar tissue.

4. The method of claim 3 wherein the depth is in the range of from about 0.5 mm to 5 mm.

5. The method of claim 1 wherein the scar treating liquid is a bio-compatible liquid substantially lacking any permanent pigmentation agents.

6. The method of claim 5 wherein the scar treating liquid is sterile distilled water.

7. The method of claim 5 wherein the scar treating liquid is a saline solution.

8. The method of claim 7 wherein the saline solution is selected-from the group consisting of normal saline, half-normal saline, quarter-normal saline, Ringer's solution, and solution of 5% dextrose in water (D5W).

9. The method of claim 1 wherein the scar treating liquid comprises a therapeutic agent for promoting wound healing.

10. The method of claim 9 wherein the therapeutic agent is selected from the group consisting of antibacterial agents, antioxidants, enzymes, growth hormones, anti-inflammatory agents, growth factors, and combinations thereof.

11. The method of claim 1 wherein the scar tissue is selected from the group consisting of keloid scars, hypertrophic scars, and combinations thereof.

12. The method of claim 1 further comprising occluding the perforated area of the skin containing the scar tissue for retaining moisture and promoting wound healing.

13. The method of claim 12 wherein the occluding step further comprises applying a coating of an ointment over the perforated area of the skin containing the scar tissue.

14. The method of claim 13 wherein the ointment further comprises a vasoconstrictor.

15. The method of claim 14 wherein the vasoconstrictor is phenylephrine.

16. The method of claim 1 wherein the perforating step further comprises forming at least one perforation in the scar tissue with at least one needle.

17. The method of claim 16 wherein the needle size of the at least one needle is less than 0.54 mm.

18. The method of claim 17 wherein the needle size is from about 0.25 mm to 0.35 mm.

19. The method of claim 16 where the at least one needle is a reciprocating needle.

20. The method of claim 19 wherein the reciprocating needle reciprocates at a rate of from about 50 to 3000 cycles per minute.

21. The method of claim 16 wherein the at least one needle is an array of needles ranging from about two to twenty needles.

22. The method of claim 16 wherein the forming step further comprises orienting the at least one needle at an angle of from about 30° to 90° to the point of perforation in the skin.

23. An apparatus for treating an area of the skin containing scar tissue, said apparatus comprising:
a needle bar comprising at least one needle, said at least one needle adapted for perforating the area of the skin containing scar tissue to yield at least one perforation;
oscillating means for oscillating the bar to axially move said at least one needle in a reciprocating motion; and
scar treating liquid supply means associated with said at least one needle for supplying a scar treating liquid to the perforation for diffusion into the scar tissue.

24. The apparatus of claim 23 wherein the needle bar has a size of from about 15- to 16-gauge.

25. The apparatus of claim 23 the at least one needle comprises an array of needles ranging from two to twenty needles.

26. The apparatus of claim 23 wherein the oscillating means comprises a reciprocating assembly operatively engaged to the needle bar.

27. The apparatus of claim 26 further comprising a needle bar holder connected to the reciprocating assembly for retaining the distal end of the needle bar to at least substantially minimize lateral movement of the needle bar.

28. The apparatus of claim 27 wherein said needle bar holder further comprises a bore for snugly receiving the distal end of the needle bar, and a set screw fastener extending through the needle bar holder for securely retaining the distal end of the needle bar in position within the bore.

29. The apparatus of claim 23 wherein the reciprocating assembly is configured to reciprocate the at least one needle at a rate of from about 50 to 3000 cycles per minute.

30. The apparatus of claim 23 wherein the needle bar is contained within a handholdable tubular housing, said scar treating liquid supply means comprises a liquid reservoir formed from a portion of said hand-holdable tubular housing, said liquid reservoir being adapted for holding and conveying the scar treating liquid to the at least one needle for supply to the perforated area of the skin.

31. The apparatus of claim 23 wherein the at least one needle is configured to form the at least one perforation to a depth of at least 0.4 mm from the surface of the scar tissue.

32. The apparatus of claim 23 wherein the scar treating liquid is a sterile bio-compatible liquid substantially lacking any permanent pigmentation agents.

33. The apparatus of claim 23 wherein the needle size of the at least one needle is less than 0.54 mm.

34. The apparatus of claim 23 wherein the scar treating liquid comprises a therapeutic agent for promoting wound healing.

35. The apparatus of claim 23 wherein the scar tissue is selected from the group consisting of keloid scars, hypertrophic scars, and combinations thereof.
